(19) United States
(12) Patent Application Publication
(10) Pub. No.: US 2007/0195969 A1
(43) Pub. Date: Aug. 23, 2007

(54) SOUND OUTPUT DEVICE AND METHOD FOR HEARING PROTECTION

(75) Inventors: Rong-Hwa Ding, Shenzhen (CN); Ho-Leung Cheung, Shenzhen (CN); Kuan-Hong Hsieh, Shenzhen (CN)

Correspondence Address: NORTH AMERICA INTELLECTUAL PROPERTY CORPORATION, P.O. BOX 506, MERRIFIELD, VA, 22116, US

(73) Assignee: HON HAI PRECISION INDUSTRY CO., LTD., Taipei Hsien (TW)

(21) Appl. No.: 11/567,186

(22) Filed: Dec. 5, 2006

(30) Foreign Application Priority Data
Feb. 18, 2006 (CN) 200610033845.6

(51) Int. Cl. A61F 11/06; H04R 1/10

(52) U.S. Cl. 381/72; 381/74

(57) ABSTRACT

The present invention relates to a hearing protection method of a sound output device, includes steps of: obtaining analog audio signals from an audio signal source via a plug of the sound output device; converting the analog audio signals to generate digital audio signals; sampling the digital audio signals to obtain a plurality of amplitude values of the sampled digital audio signals; calculating an audio energy in every predetermined time period according to the amplitude values sampled in the predetermined time period; determining whether the audio energy reaches a predetermined value; generating a hearing protect signal if the audio energy reaches the predetermined value; and in response to the hearing protect signal, automatically reducing a current gain value or outputting prompt information, thus, preventing hearing impairment of users. The present invention also provides a corresponding sound output device for hearing protection.

### BACKGROUND OF THE INVENTION

1. Field of the Invention

The present invention relates to a sound output device and method for hearing protection, especially to a sound output device and method for calculating audio energy before outputting audio signals, and automatically adjusting a gain value for amplifying the audio signals or outputting prompt signals according to the audio energy.

2. Description of Related Art

Developments in the digital technology has made portable audio devices (such as MP3 player) become popular among people. When environmental noise external of the portable audio device is loud or when a favorite song is played, a user commonly increases a volume of a portable device. However users all have a physiological hearing threshold, i.e., loudness discomfort level (LDL). If the user is exposed to an environment with a noise volume level that is larger than the user's LDL for a long time, the user's hearing may be impaired.

In order to solve the problems mentioned, there is a common gain control system and method available in the market. The gain control system and method provide a gain value limit, and disable the user to adjust the gain value beyond the gain value limit. However, the system and method is rigid and not user friendly.

Therefore, a heretofore unaddressed need exists in the industry to overcome the aforementioned deficiencies and inadequacies.

### SUMMARY OF THE INVENTION

The present invention provides a sound output device and method for hearing protection. The sound output device and method is for calculating audio energy of audio signals in every predetermined time period, and automatically reducing a gain value for amplifying the audio signals, or outputting prompt information when the audio energy reaches a predetermined value.

The sound output device includes a plug, an analog/digital converter and a processing unit. The plug connects the sound output device to an audio signal source. The analog/digital converter receives analog audio signals from the audio signal source via the plug, and converts the analog audio signals to generate digital audio signals. The processing unit includes a sampling module, a calculating module and a hearing protecting module. The sampling module receives the digital audio signals from the analog/digital converter, samples the digital audio signals by a predetermined frequency, and obtains a plurality of amplitude values of the sampled digital audio signals. The calculating module calculates an audio energy in every predetermined time period according to the amplitude values sampled in the predetermined time period. The hearing protecting module determines whether the audio energy reaches a predetermined value, and generates a hearing protect signal if the audio energy reaches the predetermined value.

The hearing protecting method includes the steps of: obtaining analog audio signals from an audio signal source via a plug of the sound output device; converting the analog audio signals to generate digital audio signals; sampling the digital audio signals to obtain a plurality of amplitude values of the sampled digital audio signals; calculating an audio energy in every predetermined time period according to the amplitude values sampled in the predetermined time period; determining whether the audio energy reaches a predetermined value; generating a hearing protect signal if the audio energy reaches the predetermined value.

Other systems, methods, features, and advantages will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

FIG. 1 is a schematic diagram of a sound output device for hearing protection in accordance with a preferred embodiment of the present invention;

FIG. 2 is a block diagram of a hardware infrastructure of the sound output device for hearing protection of FIG. 1;

FIG. 3 is a schematic diagram of main function modules of a gain managing unit of FIG. 1;

FIG. 4 is a flowchart of a preferred method for hearing protection by utilizing the sound output device of FIG. 1;

FIG. 5 is a schematic diagram of a hardware infrastructure of a sound output device for hearing protection in accordance with an alternative embodiment of the present invention; and

FIG. 6 is a schematic diagram of main function modules of a gain managing unit of FIG. 5.

### DETAILED DESCRIPTION OF THE INVENTION

In the following embodiments, for simplicity, a hearing protection function incorporated in a sound output device, such as an earphone, is depicted. The sound output device of the present invention allows hearing protection while reproducing sounds. The following detailed description of the embodiments is made with reference to the attached drawings.

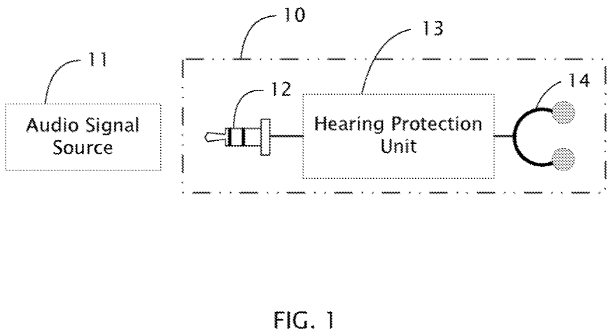

FIG. 1 is a schematic diagram of a hardware infrastructure of a sound output device in accordance with a preferred embodiment of the present invention. The sound output device 10 includes a plug 12, a hearing protection unit 13, and a sound transducer 14. The sound output device 10 receives analog audio signals from an audio signal source 11 via the plug 12, and sends the analog audio signals to the hearing protection unit 13. The audio signal source 11 can be a music player, a radio player, a TV set, and so on.

The hearing protection unit 13 converts the analog audio signals to generate digital audio signals, calculates an audio energy of the digital audio signals in every predetermined time period, and when the audio energy reaches a predetermined value, automatically reduces a current gain value, or outputs prompt information to alert a user to manually reduce the current gain value, thus, preventing hearing impairment of the user. The sound transducer 14 receives the analog audio signals sent from the audio signal source 11, and reproduces sounds corresponding to the analog audio signals. The sound transducer 14 may be an earphone or a speaker.

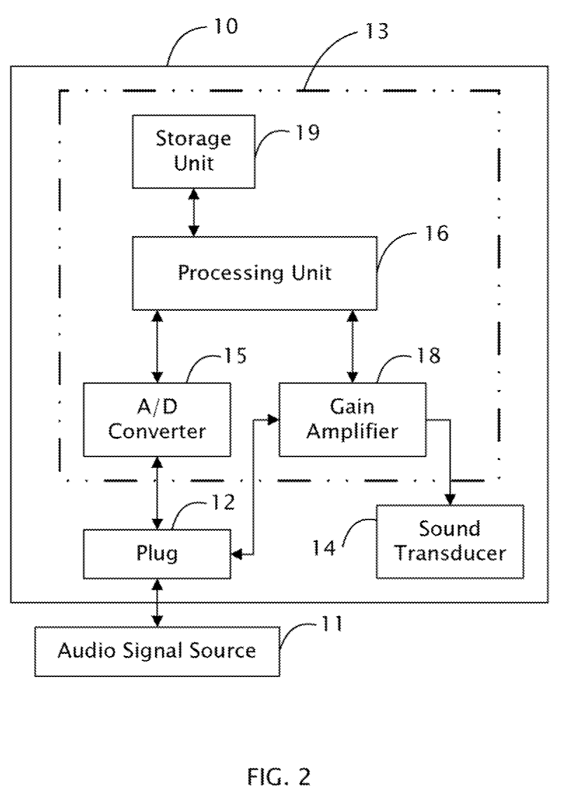

FIG. 2 is a block diagram of a hardware infrastructure of the sound output device for hearing protection. The hearing protection unit 13 includes an analog/digital (A/D) converter 15, a processing unit 16, a gain amplifier 18 and a storage unit 19. The storage unit 19 stores a default gain value. The storage unit 19 can be a flash storage, a hard disk driver, and the like. The processing unit 16 controls a plurality of elements of the hearing protection unit 13, i.e., the A/D converter 15, the gain amplifier 18 and the storage unit 19.

The A/D converter 15 receives the analog audio signals from the audio signal source 11 via the plug 12, converts the analog audio signals to the digital audio signals, and sends the digital audio signals to the processing unit 16.

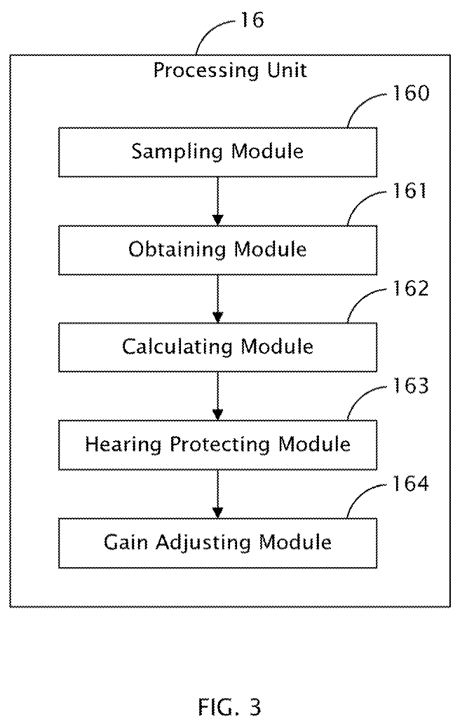

Referring to FIG. 3, the processing unit 16 includes a sampling module 160, an obtaining module 161, a calculating module 162, a hearing protection module 163, and a gain adjusting module 164. The sampling module 160 receives the digital audio signals from the A/D converter 15, samples the digital audio signals at a predetermined frequency, and obtains a plurality of amplitude values of the digital audio signals sampled in the predetermined time period.

The obtaining module 161 obtains the default gain value from the storage unit 19. The calculating module 162 calculates an audio energy in every predetermined time period by: Q=[Σ(mi*V)²/N]^{1/2}, wherein Q represents the audio energy of the digital audio signals in the predetermined time period, V represents the default gain value, mi represents the amplitude values sampled in the predetermined time period, N represents a count of the amplitude values sampled in the predetermined time period, and i is any natural number from 1 to N.

The hearing protecting module 163 determines whether the audio energy reaches the predetermined value, and if the audio energy reaches the predetermined value, generates a hearing protect signal, and sends the hearing protect signal to the gain adjusting module 164.

In response to the hearing protect signal, the gain adjusting module 164 automatically reduces the default gain value to a reduced gain value, signals the gain amplifier 18 to amplify the analog audio signals received from the audio signal source 11 by the reduced gain value, sends the amplified analog audio signal to the sound transducer 14, and replaces the default gain value stored in the storage unit 19 with the reduced gain value.

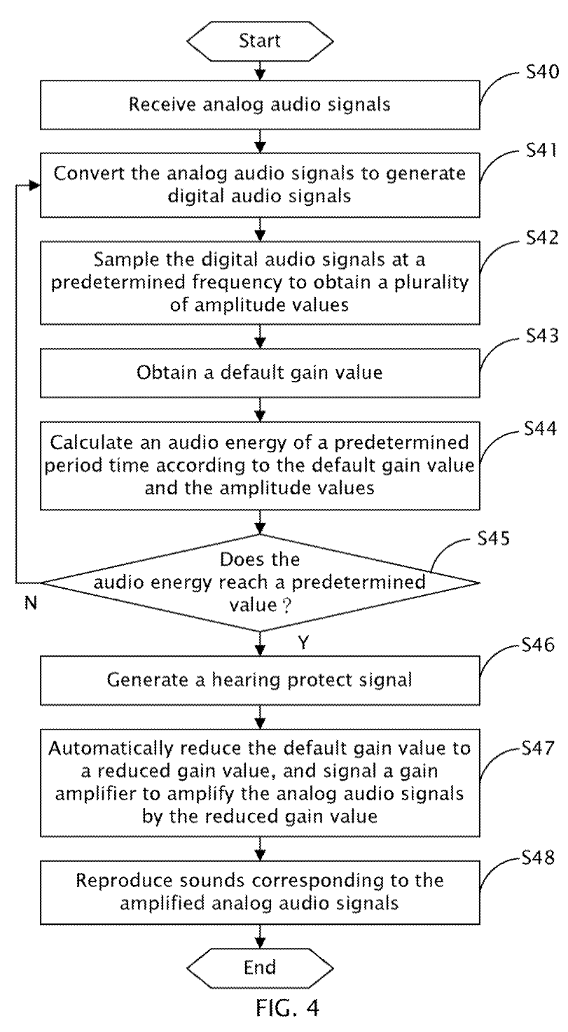

FIG. 4 is a flowchart of a preferred method for hearing protection by utilizing the sound output device of FIG. 1. In step S40, the A/D converter 15 receives analog audio signals from the audio signal source 11 via the plug 12.

In step S41, the A/D converter 15 converts the analog audio signals to generate the digital audio signals, and sends the digital audio signals to the processing unit 16.

In step S42, the sampling module 160 receives the digital audio signals from the A/D converter 15, samples the digital audio signals at the predetermined frequency, and obtaining a plurality of amplitude values of the sampled digital audio signals.

In step S43, the obtaining module 161 obtains the default gain value from the storage unit 19.

In step S44, the calculating module 161 calculates the audio energy of the digital audio signals in the predetermined time period, according to the amplitude values and the default gain value.

In step S45, the hearing protecting module 163 determines whether the audio energy reaches the predetermined value.

If the audio energy does not reach the predetermined value, the procedure goes to step S41; if the audio energy reaches the predetermined value, in step S46, the hearing protecting module 163 generates the hearing protect signal, and sends the hearing protect signal to the gain adjusting module 164.

In step S47, in response to the hearing protect signal, the gain adjusting module 164 automatically reduces the default gain value to the reduced gain value, signals the gain amplifier 18 to amplify the analog audio signals received from the audio signal source 11 by the reduced gain value, sends the amplified analog audio signal to the sound transducer 14, and replaces the default gain value stored in the storage unit 19 with the reduced gain value.

In step S48, the sound transducer 14 receives the amplified audio signal from the gain amplifier 18, and reproduces sounds corresponding to the amplified analog audio signals.

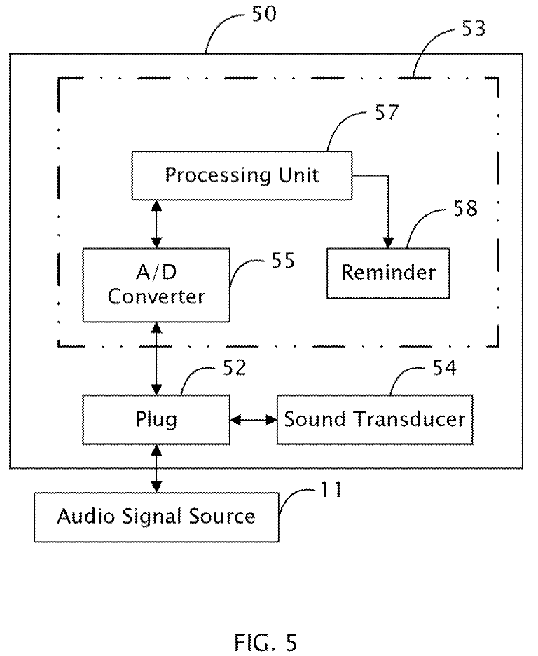

FIG. 5 is a block diagram of a hardware infrastructure of the sound output device for hearing protection in accordance with an alternative embodiment of the present invention. The sound output device 50 includes a plug 52, a hearing protection unit 53, and a sound transducer 54. The hearing protection unit 53 includes an analog/digital (A/D) converter 55, a processing unit 57 and a reminder 58. The processing unit 57 controls a plurality of elements of the hearing protection unit 53, i.e., the A/D converter 55 and the reminder 58. The reminder 58 may be an acoustical reminding device such as a buzzer, or a visual reminding device such as an LED (light-emitting diode).

The audio signal source 11 sends the analog audio signals to the sound transducer 54 and the A/D converter 55 respectively via the plug 52. The sound transducer 54 reproduces sounds corresponding to the analog audio signals.

The A/D converter 55 receives the analog audio signals from the audio signal source 11 via the plug 52, converts the analog audio signals to generate digital audio signals, and sends the digital audio signals to the processing unit 57.

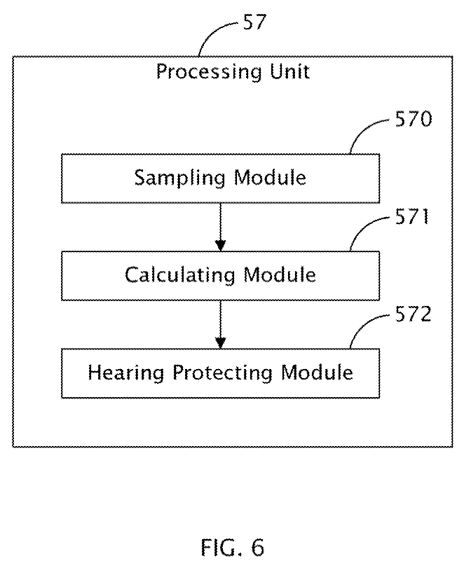

Referring to FIG. 6, the processing unit 57 includes a sampling module 570, a calculating module 571 and a hearing protecting module 572. The sampling module 570 receives the digital from the A/D converter 55, samples the digital audio signals by the predetermined frequency, and obtains a plurality of amplitude values of the sampled digital audio signals.

The calculating module 571 periodically calculates an audio energy in every predetermined time period by: Q=[Σ(mi)²/N]^{1/2}, wherein Q represents the audio energy of the digital audio signals in the predetermined time period, mi presents the amplitude values sampled in the predetermined time period, N presents a count of the amplitude values sampled in the predetermined time period, and i is any natural number from 1 to N.

The hearing protecting module 572 determines whether the audio energy reaches the predetermined value, and if the audio energy reaches the predetermined value, generates the hearing protect signal, and sends the hearing protect signal to the reminder 58. The reminder 58 receives the hearing protect signal, and outputs prompt information in response to the hearing protect signal. The prompt information includes acoustical reminding information and visual reminding information.

It is understood that the invention may be embodied in other forms without departing from the spirit thereof. Thus, the present examples and embodiments are to be considered in all respects as illustrative and not restrictive, and the invention is not to be limited to the details given herein.

What is claimed is:

1. A sound output device for hearing protection, comprising:
a plug configured for connecting to an audio signal source;
an analog/digital converter configured for receiving analog audio signals from the audio signal source via the plug, and converting the analog audio signals to generate digital audio signals;
a processing unit comprising:
a sampling module for receiving the digital audio signals from the analog/digital converter, sampling the digital audio signals by a predetermined frequency, and obtaining a plurality of amplitude values of the sampled digital audio signals;
a calculating module for calculating an audio energy in every predetermined time period according to the amplitude values sampled in the predetermined time period; and
a hearing protecting module for determining whether the audio energy reaches a predetermined value, and generating a hearing protect signal if the audio energy reaches the predetermined value.

2. The sound output device according to claim 1, further comprising a storage unit being configured for storing a default gain value, wherein the processing unit further comprises an obtaining module for obtaining the default gain value acting as a parameter for calculating the audio energy.

3. The sound output device according to claim 2, wherein the audio energy is calculated by: Q=[Σ(mi*V)²/N]^{1/2}, wherein Q represents the audio energy of the digital audio signals in the predetermined time period, V represents the default gain value, mi represents the amplitude values sampled in the predetermined time period, N represents a count of the amplitude values sampled in the predetermined time period, and i is any natural number from 1 to N.

4. The sound output device according to claim 2, further comprising a gain amplifier being configured for receiving the analog audio signals from the audio signal source via the plug, and amplifying the analog audio signals.

5. The sound output device according to claim 4, wherein the processing unit further comprises a gain adjusting module, in response to the hearing protect signal, for automatically reducing the default gain value to a reduced gain value, signaling the gain amplifier to amplify the analog audio signals by the reduced gain value, and replacing the default gain value stored in the storage unit with the reduced gain value.

6. The sound output device according to claim 5, further comprising a sound transducer for receiving the analog audio signals amplified by the gain amplifier, and reproducing sounds corresponding to the analog audio signals.

7. The sound output device according to claim 1, wherein the calculating module calculates the audio energy by: Q=[Σ(mi)²/N]^{1/2}, wherein Q represents the audio energy of the digital audio signals in the predetermined time period, mi presents the amplitude values sampled in the predetermined time period, N presents a count of the amplitude values sampled in the predetermined time period, and i is any natural number from 1 to N.

8. The sound output device according to claim 1, further comprising a reminder for receiving the hearing protect signal, and generating prompt information in response to the hearing protect signal.

9. The sound output device according to claim 8, wherein the prompt information is selected from the group consisting of visual reminding information and acoustical reminding information.

10. A hearing protection method of a sound output device, the method comprising the steps of:
obtaining analog audio signals from an audio signal source via a plug of the sound output device;
converting the analog audio signals to generate digital audio signals;
sampling the digital audio signals to obtain a plurality of amplitude values of the sampled digital audio signals;
calculating an audio energy in every predetermined time period according to the amplitude values sampled in the predetermined time period;
determining whether the audio energy reaches a predetermined value; and
generating a hearing protect signal if the audio energy reaches the predetermined value.

11. The method according to claim 10, further comprising the step of: obtaining a default gain value acting as a parameter for calculating the audio energy.

12. The method according to claim 1, wherein the audio energy is calculated by: Q=[Σ(mi*V)²/N]^{1/2}, wherein Q represents the audio energy of the digital audio signals in the predetermined time period, V represents the default gain value, mi represents the amplitude values sampled in the predetermined time period, N represents a count of the amplitude values sampled in the predetermined time period, and i is any natural number from 1 to N.

13. The method according to claim 11, further comprising the steps of: in response to the hearing protect signal, automatically reducing the default gain value to a reduced gain value, and replacing the default gain value with the reduced gain value.

14. The method according to claim 13, further comprising the steps of: amplifying the analog audio signals received from the audio signal source by the reduced gain value, and reproducing sounds corresponding to the amplified analog audio signals.

15. The method according to claim 10, wherein the audio energy is calculated by: Q=[Σ(mi)²/N]^{1/2}, wherein Q represents the audio energy of the digital audio signals in the predetermined time period, mi represents the amplitude values sampled in the predetermined time period, N represents a count of the amplitude values sampled in the predetermined time period, and i is any natural number from 1 to N.

16. The method according to claim 10, further comprising the steps of: receiving the hearing protect signal, and generating prompt information in response to the hearing protect signal.

17. The method according to claim 16, wherein the prompt information is selected from the group consisting of visual reminding information and acoustical reminding information.
